(19) United States
(12) Patent Application Publication
(10) Pub. No.: US 2023/0094123 A1
(43) Pub. Date: Mar. 30, 2023

(54) Wearable Device, Perspiration Analysis Device, and Perspiration Analysis Method

(71) Applicant: Nippon Telegraph and Telephone Corporation, Tokyo (JP)

(72) Inventors: Yuki Hashimoto, Tokyo (JP); Takako Ishihara, Tokyo (JP); Kei Kuwabara, Tokyo (JP)

(21) Appl. No.: 17/802,052

(22) PCT Filed: Mar. 4, 2020
(86) PCT No.: PCT/JP2020/009101

§ 371 Date: Aug. 24, 2022

(51) Int. Cl. A61B 5/00

(52) U.S. Cl. CPC A61B 5/4266; A61B 5/6802

(57) ABSTRACT

A wearable device includes a base material including a first surface, a first flow path being formed in the base material, including one end that opens into the first surface, and extending along a direction toward a second surface opposite to the first surface of the base material, a second flow path being formed in the base material and including one end connected to another end of the first flow path and another end that opens into the second surface, a water absorbing structure that is provided on the second surface and absorbs sweat transported from the first flow path through the second flow path and secreted from skin of the living body, and a sensor that is disposed on the base material, detects an electrical signal deriving from a predetermined component included in the sweat flowing from the first flow path to the second flow path.

### CROSS-REFERENCE TO RELATED APPLICATIONS

This application is a national phase entry of PCT Application No. PCT/JP2020/009101, filed on Mar. 4, 2020, which application is hereby incorporated herein by reference.

### TECHNICAL FIELD

The present disclosure relates to a wearable device, a perspiration analysis apparatus, and a perspiration analysis method.

### BACKGROUND

A living body such as a human body has tissues that perform electrical activities such as muscles and nerves, and in order to keep these tissues operating normally, it is provided with a mechanism for keeping an electrolyte concentration in the body constant mainly by the actions of the autonomic nervous system and the endocrine system.

For example, when a human body is exposed to a hot environment for an extended period of time, and excessive exercise or the like is taken, a large amount of moisture in the body is lost due to perspiration, and an electrolyte concentration may fall outside a normal value. In such a case, various symptoms typified by heatstroke occur in the human body. Thus, in order to recognize a dehydration condition of the body, it can be said that monitoring an amount of perspiration and an electrolyte concentration in sweat is one of beneficial techniques.

For example, in NPL 1, as a typical related art for measuring an amount of perspiration, a change in an amount of water vapor during perspiration is measured. In the technique described in NPL 1, an amount of perspiration is estimated based on a difference in humidity with respect to the outside air, and thus the air in a measurement system needs to be replaced by using an air pump.

Then, in recent years, wearable devices attached to a user are becoming widespread due to development of the ICT industry and a reduction in size and weight of a computer. The wearable devices are attracting attention for practical use in health care and fitness fields.

For example, even when a measurement technique for monitoring an amount of perspiration of a user and an electrolyte concentration in sweat is implemented by a wearable device, it is necessary to reduce the size of the device. For example, when the technique for measuring an amount of perspiration described in NPL 1 is to be implemented by a wearable device, an air pump for replacing the air in a measurement system occupies relatively large volume, and thus it can be said that a reduction in size of the entire device has a problem.

### CITATION LIST

### Non Patent Literature

NPL 1: Noriko Tsuruoka, Takahiro Kono, Tadao Matsunaga, Ryoichi Nagatomi, Yoichi Haga, “Development of Small Sweating Rate Meters and Sweating Rate Measurement during Mental Stress Load and Heat Load”, Transactions of Japanese Society for Medical and Biological Engineering, Vol. 54, No. 5, pp. 207-217, 2016.

### SUMMARY

### Technical Problem

The present disclosure has been made to solve the above-described problems, and an object thereof is to provide a wearable device that can measure a physical amount of sweat without using an air pump for replacing the air in a measurement system.

### Means for Solving the Problem

In order to solve the problem described above, a wearable device according to the present disclosure is a wearable device attached to a living body, and includes a base material including a first surface and a second surface opposite to the first surface, a first flow path being formed in the base material including one end that opens into the first surface, and extending along a direction toward the second surface, a first flow path being formed in the base material, including one end that opens into the first surface, and extending along a direction toward the second surface opposite to the first surface of the base material, a second flow path formed in the base material including one end connected to another end of the first flow path and another end that opens into the second surface, a water absorbing structure that is provided on the second surface and absorbs sweat transported from the first flow path through the second flow path and secreted from skin of the living body, and a sensor that is disposed on the base material, detects an electrical signal deriving from a predetermined component included in the sweat flowing from the first flow path to the second flow path, and outputs the electrical signal, in which a diameter of the second flow path is smaller than a diameter of the first flow path.

In order to solve the problem described above, a perspiration analysis apparatus according to the present disclosure includes the wearable device described above, a first calculation circuit that calculates, from a frequency of occurrence of a local maximum value or a local minimum value of the electrical signal output from the sensor, a physical amount related to perspiration of the living body, and an output unit configured to output the physical amount calculated and related to the perspiration.

In order to solve the problem described above, a perspiration analysis method according to the present disclosure includes causing a first flow path being formed in a base material including a first surface in contact with skin of a living body and a second surface opposite to the first surface, including one end that opens into the first surface, and extending along a direction toward the second surface to transport sweat secreted from the skin, causing a second flow path being formed in the base material, having a diameter smaller than a diameter of the first flow path, and including one end connected to another end of the first flow path and another end that opens into the second surface to transport the sweat, causing a water absorbing structure provided on the second surface to absorb the sweat transported from the first flow path through the second flow path, detecting an electrical signal deriving from a predetermined component included in the sweat flowing from the first flow path to the second flow path to output the electrical signal, calculating, from the electrical signal output in the detecting, at least any of a physical amount related to perspiration of the living body and a concentration of the predetermined component included in the sweat, and outputting a calculation result in the calculating.

### Effects of Embodiments of the Invention

The present disclosure includes the first flow path including one end that opens into the first surface of the base material and extending along the direction toward the second surface opposite to the first surface of the base material, the second flow path including one end connected to the other end of the first flow path and the other end that opens into the second surface and having a diameter smaller than a diameter of the first flow path, and a water absorbing structure that absorbs sweat transported from the first flow path through the second flow path. Thus, a physical amount related to the sweat can be measured without using an air pump for replacing the air in a measurement system.

### BRIEF DESCRIPTION OF THE DRAWINGS

FIG. 1 is a cross-sectional view of a wearable device according to an embodiment of the present disclosure.

FIG. 2 is a diagram for describing an electrical signal acquired by the wearable device according to the present embodiment.

FIG. 3A is a diagram for describing a state of sweat in a flow path corresponding to the electrical signal in FIG. 2.

FIG. 3B is a diagram for describing a state of sweat in the flow path corresponding to the electrical signal in FIG. 2.

FIG. 3C is a diagram for describing a state of sweat in the flow path corresponding to the electrical signal in FIG. 2.

FIG. 4 is a block diagram illustrating a functional configuration of a perspiration analysis apparatus including the wearable device according to the present embodiment.

FIG. 5 is a block diagram illustrating an example of a hardware configuration of the perspiration analysis apparatus including the wearable device according to the present embodiment.

FIG. 6 is a flowchart for describing an operation of the perspiration analysis apparatus including the wearable device according to the present embodiment.

### DETAILED DESCRIPTION OF ILLUSTRATIVE EMBODIMENTS

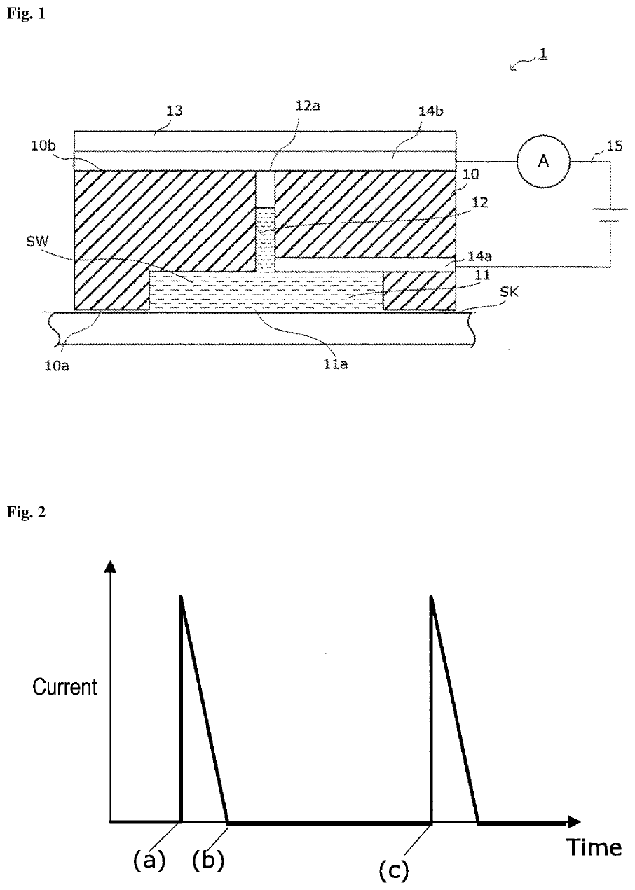

Hereinafter, preferred embodiments of the present disclosure will be described in detail with reference to FIGS. 1 to 6.

### Summary of Embodiments of the Invention

First, an outline of a wearable device 1 according to an embodiment of the present disclosure will be described with reference to FIG. 1.

FIG. 1 is a diagram schematically illustrating a cross section of the wearable device 1. The wearable device 1 includes a base material 10 attached to a user (living body) and a mechanism provided on the base material 10 for collecting sweat SW in a liquid state secreted from a sweat gland of skin SK of the user and discharging the sweat SW out of a second flow path 12 for each certain volume.

In the present embodiment, the mechanism for collecting the sweat SW and discharging the sweat SW out of the second flow path 12 includes the base material 10 including a first surface 10a disposed in contact with the skin SK of the user, a first flow path 11 being formed in the base material 10, including one end that opens into the first surface 10a, and extending along a direction toward a second surface 10b opposite to the first surface 10a of the base material 10, the second flow path 12 that is formed in the base material 10, is connected to the other end of the first flow path 11, and opens into the second surface 10b, and a water absorbing structure 13 that is provided on the second surface 10b and absorbs the sweat SW transported from the first flow path 11 through the second flow path 12 and secreted from the sweat gland of the skin SK. Further, a diameter of the second flow path is smaller than a diameter of the first flow path.

Configuration of Wearable Device

Next, the embodiment of the present disclosure will be described with reference to FIGS. 1 to 9. FIG. 1 is the schematic diagram of the cross section of the wearable device 1.

The wearable device 1 includes the base material 10 attached to the user, the first flow path 11 and the second flow path 12 that are formed in the base material 10, the water absorbing structure 13, and a sensor 15 including an electrode 14a (first electrode) and an electrode 14b (second electrode).

The base material 10 is disposed with the first surface 10a in contact with the skin SK of the user. The base material 10 includes the second surface 10b opposite to the first surface 10a. The second surface 10b is a surface of the base material 10 formed in a position farther from the skin SK. The base material 10 has an external shape of a cuboid, for example. As a material of the base material 10, a non-conductive or conductive resin, an alloy, or the like can be used, and in the present embodiment, a case in which a non-conductive material is used is described as an example.

The first flow path 11 is formed in the base material 10, includes one end that opens into the first surface 10a, and extends along the direction toward the second surface 10b of the base material 10. The one end of the first flow path 11 forms an opening 11a in the first surface 10a. The other end of the first flow path 11 is connected to the second flow path 12 described below.

The opening 11a formed in the first surface 10a proximate to the one end of the first flow path 11 is disposed in contact with the skin SK, and the sweat SW is collected from the opening 11a. When the sweat SW is continuously secreted from the sweat gland of the skin SK, a water level of the liquid sweat SW reaches the other end of the first flow path 11. As illustrated in FIG. 1, the first flow path 11 has a circular or rectangular cross-sectional shape, for example. Further, the first flow path 11 can be formed to have a flow path width greater than a flow path length.

The second flow path 12 is formed in the base material 10 and includes one end connected to the other end of the first flow path 11 and the other end that opens into the second surface 10b of the base material 10. An opening 12a formed by the second flow path 12 penetrating through the base material 10 is formed in the second surface 10b. Further, as illustrated in FIG. 1, a diameter of the second flow path 12 is sufficiently smaller than a diameter of the first flow path 11. For example, the second flow path includes a thin tube and has a cross-sectional area of approximately 1 mm², or 1 mm² or less. A cross-sectional shape of the second flow path 12 can be, for example, circular, rectangular, or the like. Further, a flow path length of the second flow path 12 may be formed so as to be longer than a flow path length of the first flow path 11.

In the present embodiment, as illustrated in FIG. 1, by using the second flow path 12 having a cross-sectional area sufficiently smaller than a cross-sectional area of the first flow path 11, the sweat SW can be transported from the first flow path 11 to the second flow path 12 by further using a capillary phenomenon in addition to osmotic pressure of the sweat SW secreted from the sweat gland. Note that an inner wall of the second flow path 12 may be subjected to surface treatment with a material having high wettability with respect to the sweat SW.

The water absorbing structure 13 is provided on the second surface 10b of the base material 10, and absorbs the sweat SW transported from the first flow path 11 to the second flow path 12. More specifically, the water absorbing structure 13 is disposed in contact with the opening 12a formed in one end of the second flow path 12. The water absorbing structure 13 absorbs, from a contact area with the opening 12a, the sweat SW transported from the first flow path 11 through the second flow path 12.

The water absorbing structure 13 can be achieved by fibers such as cotton and silk, a porous ceramic board, a hydrophilic flow path, and the like. Further, the water absorbing structure 13 can have, for example, a rectangular sheet-like or plate-like shape corresponding to a shape of the second surface 10b of the base material 10 and covers the opening 12a being an outlet of the second flow path 12.

The sensor 15 is disposed on the base material 10, detects an electrical signal deriving from a predetermined component included in the sweat SW flowing from the first flow path 11 to the second flow path 12, and outputs the electrical signal. The sensor 15 includes the pair of electrodes 14a and 14b and a current meter that detects energization between the electrodes 14a and 14b. The sensor 15 may include a direct current power supply. Alternatively, the electrodes 14a and 14b are formed of materials having different standard electrode potentials, and thus an electromotive force can also be generated.

The electrode 14a is disposed so as to be exposed to the first flow path 11, and the other electrode 14b is disposed so as to face the electrode 14a and be exposed to the second flow path 12, for example. Note that the electrode 14a may be disposed on the inner wall of the second flow path 12, for example, as long as the electrode 14a is spaced apart from the electrode 14b so as not to be in contact with each other.

The electrode 14b has a porous structure that transmits the sweat SW. The electrode 14b is disposed between the second surface 10b of the base material 10 in which the opening 12a being the outlet of the second flow path 12 is formed and the water absorbing structure 13.

The electrode 14b can use, for example, a mesh electrode as the porous structure. For example, the mesh electrode can be achieved by a porous metal thin film formed by a plating technique on a surface of the water absorbing structure 13. Alternatively, the mesh electrode can also be achieved by a conductive polymer impregnated in the fibers of the water absorbing structure 13. Alternatively, the mesh electrode in which fibers coated with metal by vapor deposition or the like are woven into the water absorbing structure 13 can also be used. Note that a flow path structure may be used as the porous structure of the electrode 14b.

As illustrated in FIG. 1, wiring is connected to each of the electrodes 14a and 14b disposed in the first flow path 11 and the second flow path 12, respectively. Further, in the example in FIG. 1, the electrodes 14a and 14b, the current meter, and the direct current power supply are connected in series. Note that, as described above, when a configuration in which an electromotive force is generated by selecting materials of the electrodes 14a and 14b is adopted, an external power supply is omitted.

The sweat SW secreted from the sweat gland of the skin SK flows in from the first flow path 11 and is transported to the second flow path 12, and, when an amount of perspiration further increases or perspiration further continues, the liquid sweat SW reaches the opening 12a being the outlet of the second flow path 12. Furthermore, when the sweat SW comes into contact with the electrode 14b, an electrolyte such as sodium ions and potassium ions included in the sweat SW causes energization, and a current flows. When the droplet of the sweat SW is absorbed by the water absorbing structure 13, the electrode 14b is in contact with only the air, and no current flows. The sensor 15 measures a current signal detected by the current meter, and outputs the current signal.

In the wearable device 1 described above, for example, the first flow path 11 and the second flow path 12 are formed in the cuboid base material 10, a hole into which the electrode 14a is inserted is then formed in the base material 10, and the electrode 14a is inserted. Finally, the surface of the water absorbing structure 13 on which the electrode 14b being the mesh electrode is formed is bonded to the second surface 10b of the base material 10, and thus the wearable device 1 can be acquired.

As illustrated in FIG. 3A, when the sweat SW is secreted from the sweat gland of the skin SK, the sweat SW flows in from the opening 11a being an inlet of the first flow path 11 to the first flow path 11. Furthermore, the sweat SW flows into the second flow path 12 and reaches the opening 12a being the outlet of the second flow path 12. Then, as illustrated in FIG. 3B, the water absorbing structure 13 absorbs the sweat SW retained in the second flow path 12. Once the sweat SW retained in the second flow path 12 is absorbed by the water absorbing structure 13, the sweat SW is not present in the second flow path 12.

Subsequently, when the sweat SW secreted from the sweat gland of the skin SK of the user is increased again or continuously secreted, as illustrated in FIG. 3C, the sweat SW is transported into the second flow path 12. Then, the sweat SW comes into contact with the water absorbing structure 13 again, and the sweat SW having volume of the second flow path 12 is absorbed. In this way, a cycle of appearance and disappearance of the sweat SW in the second flow path 12 is repeated on a cycle synchronized with a perspiration rate in accordance with the secretion of the sweat SW.

Functional Blocks of Perspiration Analysis Apparatus

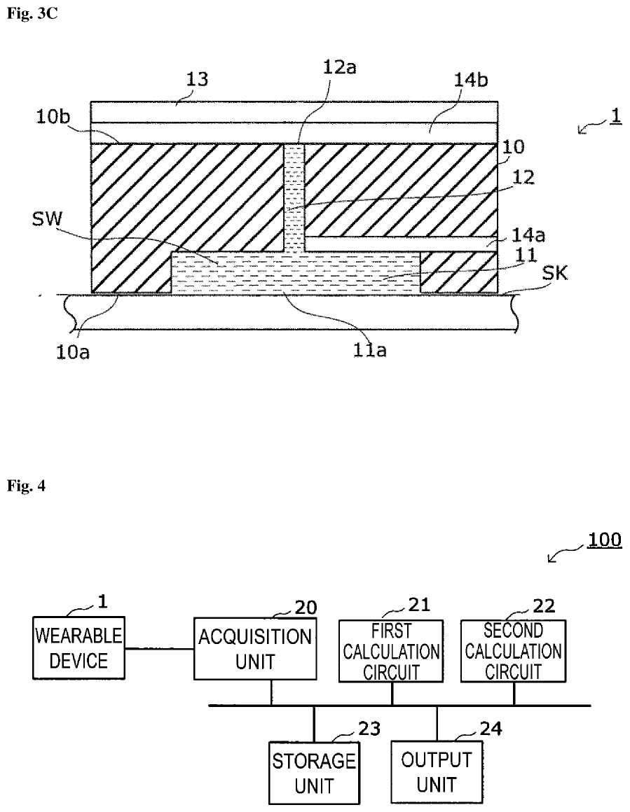

Next, a functional configuration of the perspiration analysis apparatus 100 including the wearable device 1 described above will be described with reference to a block diagram in FIG. 4.

The perspiration analysis apparatus 100 includes the wearable device 1, an acquisition unit 20, a first calculation circuit 21, a second calculation circuit 22, a storage unit 23, and an output unit 24.

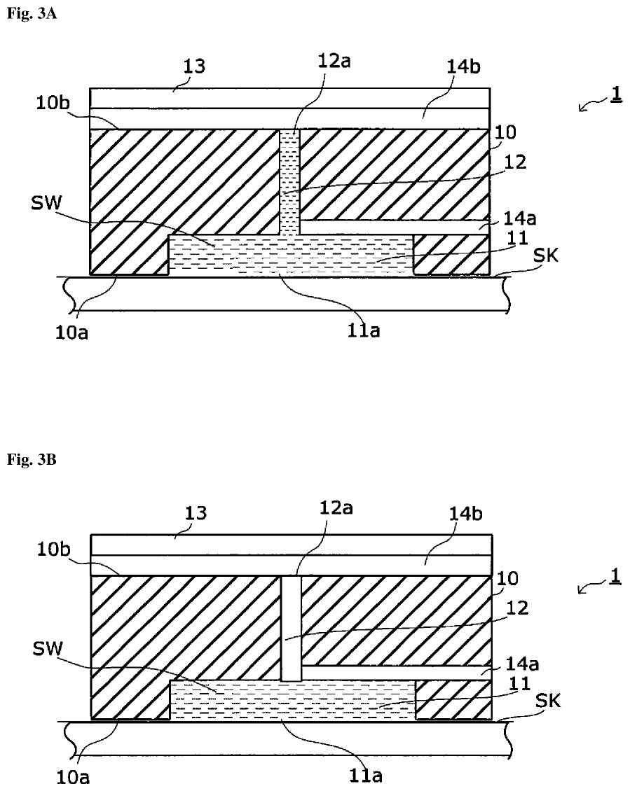

The acquisition unit 20 acquires an electrical signal acquired by the wearable device 1. The acquisition unit 20 performs signal processing, such as amplification, noise removal, and AD conversion of the acquired electrical signal. Time-series data of the acquired electrical signal is accumulated in the storage unit 23. As illustrated in FIG. 2, for example, the time-series data of the electrical signal acquired by the acquisition unit 20 is a waveform having a peak in accordance with the cycle of the appearance and disappearance of the sweat SW in the second flow path 12 described above.

FIG. 2 is an example of a current value (electrical signal) that is a physical amount related to the sweat SW electrically measured by the wearable device 1 by the sensor 15 including the electrodes 14a and 14b.

A vertical axis in FIG. 2 indicates a current value between the electrodes 14a and 14b measured by the sensor 15, and a horizontal axis indicates time. FIGS. 3A, 3B, and 3C illustrate states of the sweat SW flowing through the second flow path 12 at each time (a), (b), and (c) in FIG. 2.

Time-series data of the electrical signal in FIG. 2 has a waveform such as a periodic pulse waveform. At the time (a) illustrated in FIG. 2, as illustrated in FIG. 3A, in the wearable device 1, the sweat SW is transported to the second flow path 12, a water level of the sweat SW rises over time, and the sweat SW reaches the opening 12a being the outlet of the second flow path 12 and comes into contact with the electrode 14b formed of the mesh electrode. When the sweat SW comes into contact with the electrode 14b, the electrodes 14a and 14b are energized, and a current begins to flow.

Subsequently, at the time (b) in FIG. 2, as illustrated in FIG. 3B, the sweat SW retained in the second flow path 12 is absorbed by the water absorbing structure 13 through the mesh electrode (electrode 14b), and the electrode 14b is in contact with only the air of the second flow path 12, and thus no current flows.

At the time (c) in FIG. 2, as illustrated in FIG. 3C, as the sweat SW secreted from the sweat gland of the skin SK of the user increases, a droplet of the sweat SW is transported again from the first flow path 11 to the second flow path 12 over a certain period of time, and when the droplet comes into contact with the electrode 14b, the electrodes 14a and 14b are energized again, and a current flows.

Referring back to FIG. 4, the first calculation circuit 21 calculates a physical amount related to perspiration from a frequency of occurrence of a local maximum value or a local minimum value of the electrical signal. For example, the first calculation circuit 21 calculates, from the time-series data of the electrical signal, an amount of perspiration by multiplying predetermined volume of the second flow path 12 by the number of times of energization (the number of peaks in FIG. 2).

Further, the first calculation circuit 21 calculates a perspiration rate per unit area by dividing volume of the second flow path 12 by an energization cycle and an area of the skin SK in contact with the opening 11a being the inlet of the first flow path 11. Note that a cross-sectional area of the opening 11a can be used as an area of the skin SK.

The second calculation circuit 22 calculates a concentration of a predetermined component included in the sweat SW from a local maximum value or a local minimum value of the electrical signal acquired by the wearable device 1. For example, the second calculation circuit 22 calculates a concentration of an electrolyte such as sodium ions and potassium ions among components (water, sodium chloride, urea, lactic acid, and the like) included in the sweat SW. More specifically, the second calculation circuit 22 calculates, from an applied voltage between the electrodes 14a and 14b and a current value during energization, an average resistance value (conductivity) that depends on a concentration of an electrolyte included in the sweat SW.

The storage unit 23 stores time-series data of the electrical signal acquired from the wearable device 1 by the acquisition unit 20. In the storage unit 23, volume of the second flow path 12 and a value of a voltage applied between the electrodes 14a and 14b are previously stored.

The output unit 24 outputs the amount of perspiration, the perspiration rate, and the component concentration of the sweat SW calculated by the first calculation circuit 21 and the second calculation circuit 22. The output unit 24 can display a calculation result on a display device (not illustrated), for example. Alternatively, the output unit 24 may send a calculation result to an external communication terminal device (not illustrated) by a communication I/F 105 described below.

Hardware Configuration of Perspiration Analysis Apparatus

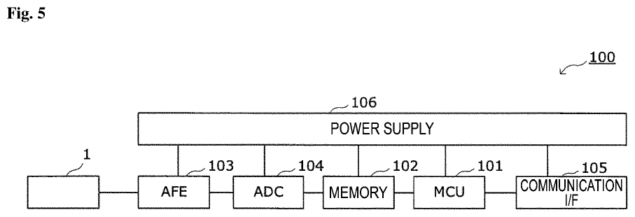

Next, an example of a hardware configuration that implements the perspiration analysis apparatus 100 including the wearable device 1 having the above-described functions will be described with reference to FIG. 5.

As illustrated in FIG. 5, for example, the perspiration analysis apparatus 100 can be implemented by a computer including an MCU 101, a memory 102, an AFE 103, an ADC 104, and a communication I/F 105 connected to each other through a bus and a program for controlling these hardware resources. In the perspiration analysis apparatus 100, for example, the wearable device 1 provided outside is connected through the bus. Further, the perspiration analysis apparatus 100 includes a power supply 106 and supplies power to the entire device other than the wearable device 1 illustrated in FIGS. 4 and 5.

A program causing the micro control unit (MCU) 101 to perform various controls or calculations is previously stored in the memory 102. Each function of the perspiration analysis apparatus 100 including the acquisition unit 20, the first calculation circuit 21, and the second calculation circuit 22 illustrated in FIG. 4 is implemented by the MCU 101 and the memory 102.

The analog front end (AFE) 103 is a circuit that amplifies a measurement signal that is a weak electrical signal representing an analog current value measured by the wearable device 1.

The analog-to-digital converter (ADC) 104 is a circuit that converts an analog signal amplified by the AFE 103 into a digital signal at a predetermined sampling frequency. The AFE 103 and the ADC 104 implement the acquisition unit 20 in FIG. 4.

The memory 102 is implemented by a non-volatile memory such as a flash memory, a volatile memory such as a DRAM, and the like. The memory 102 temporarily stores time-series data of signals output from the ADC 104. The memory 102 implements the storage unit 23 in FIG. 4.

The memory 102 includes a program storage area in which a program used by the perspiration analysis apparatus 100 to perform perspiration analysis processing is stored. Further, for example, it may have a backup area for backing up the above-described data, programs, or the like.

The communication I/F 105 is an interface circuit for communicating with various external electronic devices through a communication network NW.

For example, a communication interface compatible with a wired or wireless data communication standard such as LTE, 3G, 4G, 5G, Bluetooth (trade name), Bluetooth Low Energy, and Ethernet (trade name) and an antenna are used as the communication I/F 105. The output unit 24 in FIG. 4 is implemented by the communication I/F 105.

Note that the perspiration analysis apparatus 100 acquires time information from a clock incorporated in the MCU 101 or a time server (not illustrated) and uses the time information as sampling time.

Perspiration Analysis Method

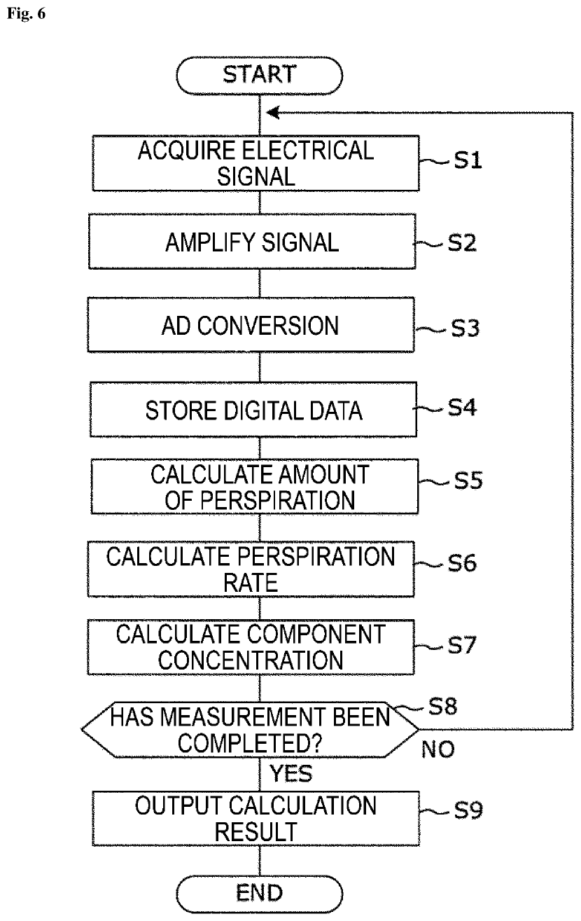

Next, an operation of the perspiration analysis apparatus 100 including the wearable device 1 having the above-described configuration will be described with reference to a flowchart in FIG. 6. When the wearable device 1 is previously attached to the user, the power supply 106 is turned on, and the perspiration analysis apparatus 100 is activated, the following processing operations are performed.

First, the acquisition unit 20 acquires an electrical signal indicating a current value from the wearable device 1 (step S1). Next, the acquisition unit 20 amplifies the electrical signal (step S2). More specifically, the AFE 103 amplifies a weak current signal measured by the wearable device 1.

Next, the acquisition unit 20 performs AD conversion on the electrical signal amplified in step S2 (step S3). Specifically, the ADC 104 converts an analog signal amplified by the AFE 103 into a digital signal at a predetermined sampling frequency. Time-series data of the electrical signal converted into the digital signal is stored in the storage unit 23 (step S4).

Next, the first calculation circuit 21 calculates an amount of perspiration of the user from a frequency of occurrence of a local maximum value of the acquired electrical signal (step S5). Subsequently, the first calculation circuit 21 calculates a perspiration rate from the frequency of occurrence of the local maximum value of the electrical signal (step S6).

Next, the second calculation circuit 22 calculates a concentration of a predetermined component included in the sweat SW from the local maximum value of the acquired electrical signal (step S7). Subsequently, when the measurement has been completed (step S8: YES), the output unit 24 outputs a calculation result including the amount of perspiration, the perspiration rate, and the component concentration (step S9). On the other hand, when the measurement has not been completed (step S8: NO), the processing returns to step S1.

Note that the first calculation circuit 21 may be configured to calculate either the amount of perspiration or the perspiration rate. The first calculation circuit 21 can also be configured, by setting, to calculate any one or two values of the amount of perspiration, the perspiration rate, and the component concentration, and an order in which the values are calculated is optional.

As described above, according to the present embodiment, in the wearable device 1, the second flow path 12 having a diameter smaller than that of the first flow path 11 is formed in the base material 10, and the sweat SW is transported from the first flow path 11 to the second flow path 12 due to perspiration. Then, when the sweat SW comes into contact with the water absorbing structure 13 provided on the opening 12a being the outlet of the second flow path 12, the sweat SW having volume of the second flow path 12 is absorbed by the water absorbing structure 13. Thus, the wearable device 1 can measure a physical amount related to sweat without using an air pump. Further, the wearable device 1 can measure, from the measured physical amount related to the sweat, a physical amount related to perspiration such as an amount of perspiration and a perspiration rate, and a component included in the sweat.

The wearable device 1 according to the present embodiment collects the sweat SW in a liquid state without using an air pump, and discharges the sweat SW from the second flow path 12 for each certain volume, and thus the size of the wearable device 1 can be made smaller. Further, as a result, the size of the perspiration analysis apparatus 100 can be reduced.

Further, the wearable device 1 according to the present embodiment includes the sensor 15 including the pair of electrodes 14a and 14b and measures time-series data of a current signal due to energization in accordance with a cycle in which the sweat SW appears in the second flow path 12 and is absorbed by the water absorbing structure 13 on a certain cycle. Thus, the wearable device 1 attached to a user can electrically measure a physical amount related to sweat.

Although the embodiments of the wearable device, the perspiration analysis apparatus, and the perspiration analysis method according to the present disclosure have been described above, the present disclosure is not limited to the above-described embodiments and can be modified into various forms that can be conceived by a person skilled in the art within the scope of the disclosure described in the aspects.

### REFERENCE SIGNS LIST

1 Wearable device
10 Base material
10a First surface
10b Second surface
11 First flow path
11a, 12a Opening
12 Second flow path
13 Water absorbing structure
14a, 14b Electrode
15 Sensor
20 Acquisition unit
21 First calculation circuit
22 Second calculation circuit
23 Storage unit
24 Output unit
100 Perspiration analysis apparatus
101 MCU
102 Memory
103 AFE
104 ADC
105 Communication I/F
106 Power supply
SW Sweat
SK Skin.

1-6. (canceled)

7. A wearable device, comprising:
a base material including a first surface and a second surface opposite to the first surface;
a first flow path in the base material, the first flow path comprising a first end that opens onto the first surface, and the first flow path extending along a direction toward the second surface;
a second flow path in the base material, the second flow path including one end connected to a second end of the first flow path and a second end that opens onto the second surface, wherein a diameter of the second flow path is smaller than a diameter of the first flow path;
a water absorbing structure on the second surface and configured to absorb sweat transported from the first flow path through the second flow path, the sweat being secreted from skin of a living body; and
a sensor on the base material and configured to detect an electrical signal derived from a predetermined component included in the sweat flowing from the first flow path to the second flow path, the sensor being further configured to output the electrical signal.

8. The wearable device according to claim 7, wherein the sensor includes:
a first electrode exposed to the first flow path, and
a second electrode spaced apart from the first electrode and exposed to the second flow path.

9. The wearable device according to claim 8, wherein:
the second electrode includes a porous body, and
the second electrode is disposed between the second surface of the base material where the second flow path opens and the water absorbing structure.

10. The wearable device according to claim 7, wherein the predetermined component is an electrolyte.

11. A perspiration analysis apparatus, comprising:
a wearable device configured to be worn by a living body, the wearable device comprising:
a base material including a first surface and a second surface opposite to the first surface;
a first flow path in the base material, the first flow path comprising a first end that opens onto the first surface, and the first flow path extending along a direction toward the second surface;
a second flow path in the base material, the second flow path including one end connected to a second end of the first flow path and a second end that opens onto the second surface, wherein a diameter of the second flow path is smaller than a diameter of the first flow path;
a water absorbing structure on the second surface and configured to absorb sweat transported from the first flow path through the second flow path, the sweat being secreted from skin of a living body; and
a sensor on the base material and configured to detect an electrical signal derived from a predetermined component included in the sweat flowing from the first flow path to the second flow path, the sensor being further configured to output the electrical signal;
a first calculation circuit configured to calculate, from a frequency of occurrence of a local maximum value or a local minimum value of the electrical signal output from the sensor, a physical amount related to perspiration of the living body; and
an output device configured to output the physical amount calculated by the first calculation circuit that is related to the perspiration of the living body.

12. The perspiration analysis apparatus according to claim 11, further comprising:
a second calculation circuit configured to calculate, from the local maximum value or the local minimum value of the electrical signal output from the sensor, a concentration of a predetermined component included in the sweat, wherein the output device is configured to output the concentration calculated by the second calculation circuit.

13. The perspiration analysis apparatus according to claim 11, wherein the sensor further includes:
a first electrode exposed to the first flow path, and
a second electrode spaced apart from the first electrode and exposed to the second flow path.

14. The perspiration analysis apparatus according to claim 13, wherein:
the second electrode includes a porous body, and
the second electrode is disposed between the second surface of the base material where the second flow path opens and the water absorbing structure.

15. The perspiration analysis apparatus according to claim 11, wherein the predetermined component is an electrolyte.

16. A perspiration analysis method, comprising:
flowing sweat secreted from skin of a living body through a first flow path, the first flow path being disposed in a base material including a first surface in contact with the skin and a second surface opposite to the first surface, the first flow path including a first end that opens onto the first surface, and the first flow path extending along a direction toward the second surface;
flowing the sweat through a second flow path in the base material, the second flow path having a diameter smaller than a diameter of the first flow path, and the second flow path including a first end connected to a second end of the first flow path and a second end that opens onto the second surface;
absorbing the sweat transported from the first flow path through the second flow path with a water absorbing structure on the second surface;
detecting an electrical signal from a predetermined component included in the sweat flowing from the first flow path to the second flow path;
calculating, from the electrical signal, at least any of a physical amount related to perspiration of the living body and a concentration of the predetermined component included in the sweat; and
outputting a calculation result of the calculating.

17. The perspiration analysis method according to claim 16, wherein the predetermined component is an electrolyte.
